(19) United States
(12) Patent Application Publication
(10) Pub. No.: US 2026/0013978 A1
(43) Pub. Date: Jan. 15, 2026

(54) SYSTEM AND METHOD FOR DESIGNING AND FABRICATING IDEALIZED IMPLANTS FOR HOLLOWED ANATOMICAL STRUCTURES

(71) Applicants: Meghan Brar, Mississauga (CA); Alex zur Linden, Elmira (CA)

(72) Inventors: Meghan Brar, Mississauga (CA); Alex zur Linden, Elmira (CA)

(21) Appl. No.: 19/264,584

(22) Filed: Jul. 9, 2025

Related U.S. Application Data
(60) Provisional application No. 63/669,143, filed on Jul. 9, 2024.

(51) Int. Cl. A61F 2/04; G16H 50/70

(52) U.S. Cl. CPC A61F 2/04; G16H 50/70; A61F 2240/002; A61F 2250/0081

(57) ABSTRACT

Described is a system and method for designing and fabricating idealized implants any hollowed anatomical structures. It includes a system and a method for obtaining and segmenting three-dimensional image data to segment an area of interest for a plurality of subjects in a population. The system and method use cross-sectional areas calculated at a plurality of discrete or relative locations to determine the relative shape of a hollowed anatomical structure, and statistical methods to find an optimal subject in a population. An idealized implant is then produced and manufactured based on the optimal subject model for a population.

### FIELD

The present specification relates generally to implants and methods of designing and fabricating implants for anatomical structures, and more specifically relates a system and method for designing and fabricating idealized stents and scaffolds for hollowed anatomical structures based on a candidate structure model.

### BACKGROUND

Lesions of anatomical structures can occur as a result of congenital, acquired, or progressive disease types, such as neoplastic disease, inflammation, cystic masses, foreign bodies, strictures, degenerative changes, or physical trauma to the body. Examples of this include congenital heart conditions, and obstructions from tumours. Tissue damage within lesion areas can impact the morphology and function of anatomical structures in varying severity, often impacting quality of life in patients. The impairment of regular function to vital hollowed structures (e.g., heart valves) leads to debilitating symptoms and life-threatening conditions in patients. Interventional treatment with medical implants is a less invasive approach to surgical resection. Interventional treatment with medical implants can be used alternatively to, or in conjunction with surgical resection, where the implants function to stabilize the lumen of the anatomical structure and enable tissue healing.

In an example of an airway, generic and patient-specific airway implants are typical interventions that are commercially available for respiratory obstructive diseases. While generic implants are far more affordable than patient-specific options, they conform poorly to the airway in comparison. According to medical literature, poor fit is linked to a high prevalence of complications, including migration, infection, and fistula development, ultimately leading to a poor prognosis. Patient-specific implants offer a curated fit, thereby diminishing complications and suggesting promising outcomes. However, patient-specific medical implants, while tailored to individual patient anatomy, are often prohibitively expensive due to the advanced imaging, design, and manufacturing processes required, making them inaccessible to many patients.

Currently, patients much choose between high expense and long wait times, or poor fit which can lead to complications, or even failure of the intervention. Idealized implants are a potential solution that can provide a curated fit to an anatomical structure and are affordable and rapid to manufacture.

However, other proposed methods for creating idealized implants for anatomical structures are limited to tubular structures, and do not apply generally to non-tubular structures or structures that are not open at one or either end, such as a cavity (e.g., heart, lungs, etc.). As such, the current methods are limited in scope, and many patients are still limited to generic or patient-specific implant methods. Further, other proposed methods to enhance stock stents are specific to tubular structures and stenting applications, and do not apply to scaffolds which include biological structures that maintain support and enhance tissue contour.

Accordingly, there remains a need for improvements in the art.

### SUMMARY

A computing system for generating implant designs, comprising: a processor; and a non-transitory memory storing an idealized implant application comprising a segmentation engine, a cross-sectioning engine, and idealization engine, and a manufacturing engine, that, when executed by the processor, cause the processor to perform acts comprising: obtaining a plurality of images captured from a subject population with a normal hollowed anatomical structure via an input device; segmenting at least one region of interest in each of the plurality of images to provide a three-dimensional model of an area of interest via segmentation software that provides data; selecting a plurality of locations within each three-dimensional model of an area of interest and generating a corresponding plurality of cross-sectional areas for the plurality of locations via a graphic user interface; determining population data from a plurality of data points that represent the relative shape of the hollowed anatomical structure for each subject in a population by determining the ratio of the cross-sectional area at one location to a different location, repeated for all possible location combinations, wherein each location combination represents a data category; discovering an optimal subject in a population from population data by statistical methods; and utilizing a model generator to refine a three-dimensional model of the optimal subject and design a implant shape corresponding to the optimal subject in a population.

A method for designing and manufacturing implants, the method comprising: obtaining a plurality of images captured from a subject population with a normal hollowed anatomical structure; segmenting at least one region of interest in each of the plurality of images to provide a three-dimensional model via segmentation software that provides data; selecting a plurality of locations within each three-dimensional model of a hollowed anatomical structure and generating a corresponding plurality of cross-sectional areas for the plurality of locations via a graphic user interface; determining population data from a plurality of data points that represent the relative shape of the hollowed anatomical structure for each subject in a population, wherein each location combination represents a data category; discovering an optimal subject in a population from population data by statistical methods; utilizing a model generator to refine and construct an idealized implant shape corresponding to the optimal subject in a population; and manufacturing the idealized implant shape.

### BRIEF DESCRIPTION OF THE DRAWINGS

FIG. 1 is a schematic of the process of designing and manufacturing an idealized implant, according to an embodiment of the present invention.

FIG. 2 is a schematic of the computing system for designing and manufacturing an idealized implant, according to an embodiment of the present invention.

FIG. 3 is a schematic of the idealized implant application, according to an embodiment of the present invention.

FIG. 4 is a left side perspective view of a candidate canine nasopharyngeal model segmented from the nasopharynx of the optimal subject, according to an embodiment of the present invention.

FIG. 5 is a left side perspective view of a candidate canine nasopharyngeal model after refinement, according to an embodiment of the present invention.

FIG. 6 is a left side perspective view of an idealized implant for a canine nasopharynx, according to an embodiment of the present invention.

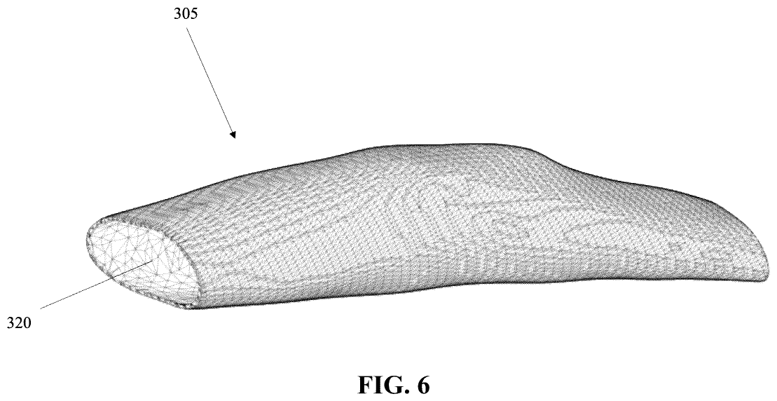

FIG. 7 is a dorsal perspective view of a candidate non-tubular structure model segmented from a subject, according to an embodiment of the present invention.

FIG. 8 is a dorsal perspective view of a candidate non-tubular structure model after refinement, according to an embodiment of the present invention.

FIG. 9 is a dorsal perspective view of the cross-sectional areas collected at a plurality of locations from one end to the other of an anatomical structure, according to an embodiment of the present invention.

FIG. 10 is a left side perspective view of an idealized implant for a non-tubular structure that is cropped to a specific area of the structure, according to an embodiment of the present invention.

### DETAILED DESCRIPTION OF THE EMBODIMENTS

The description that follows, and the embodiments described therein, are provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and, in some instances, proportions may have been exaggerated in order to more clearly depict certain features of the invention.

As used herein a “hollowed anatomical structure” refers to any part of the body that has a lumen. This definition is not limited by morphology and/or shape, but rather it is characterized as being capable of containing a substance, wherein a substance includes but is not limited to liquids and/or vapors. A “hollowed anatomical structure” as used herein is alternatively referred to as an “anatomical structure” or “structure”. The method for fabricating an idealized implant is executed by an individual referred to as the “user” which implies a person who is at least partially responsible for developing the idealized implant and includes, but is not limited to: students, clinicians, engineers and technicians. As used herein, the term “patient” can refer, interchangeably, to any human or animal. Further, as used herein, the terms “engine”, “application”, and “system” are intended to encompass computer-readable data storage that is configured with computer-executable instructions that cause certain functionality to be performed when executed by a processor.

Described herein are various features pertaining to generating optimized implant designs for treatment of hollowed anatomical structures. The various features are understood with at least the following examples.

Relevant prior art describes the use of stents for “obstructive diseases” (strictures) in a tubular structure made of non-biological materials (polymers or metal). In contrast, the present invention offers an idealized implant which can include both “stents” and “scaffolds”, where a scaffold may be used for “internal lesions” (any tissue damage which affects structure and function) in any hollowed structure. Stents are normally enclosed tubes, whereas scaffolds are more general, and can be enclosed, or can be a part of a structure (e.g., a cardiac patch). A scaffold also utilizes biological materials such as biopolymers to help heal the tissue. Stents can only provide structural support, while scaffolds can have tissue healing properties. Unlike prior art which discloses methods for constructing stents for tubular structures, the present invention refers to the application of idealized implants for any hollowed anatomical structure (e.g., hollow organs, chambers, cavities, vessels, and airways), to develop scaffolds which include biological structures that contour the anatomy and support tissue healing. The present invention refers to the design and construction of both stents made from artificial material, and scaffolds made of biological material. The methods and systems described herein can be utilized for any hollowed anatomical structure, including both tubular and non-tubular structures.

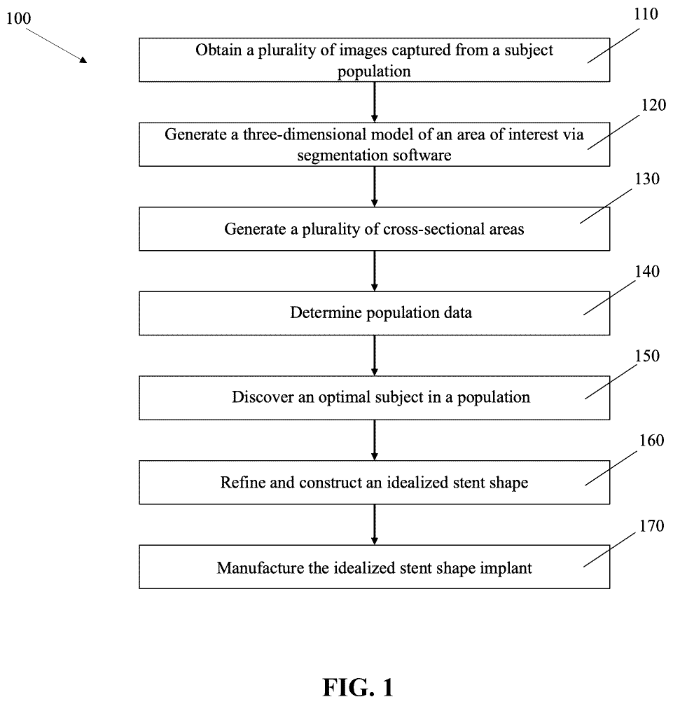

An idealized implant is a medical device that is shaped based on the average relative shape of a hollow anatomical structure in a population and functions to stabilize the anatomical structure, thereby allowing it to heal. A “population” includes, but is not limited to, species, age, gender, race, skull types, breeds, etc. and can be either humans or animals. According to an embodiment as shown in FIG. 1, a method 100 for designing and fabricating an idealized implant for a hollowed anatomical structure comprises obtaining a plurality of images captured from a subject population at step 110, generating a three-dimensional model of an area of interest via segmentation software at step 120, generating a plurality of cross-sectional areas at step 130, determining population data at step 140, discovering an optimal subject in a population at step 150, refining and constructing an idealized implant shape at step 160, and manufacturing the idealized implant shape at step 170.

At step 110, images can be generated via a three-dimensional medical imaging modality, such as computed tomography (CT) scan data or any other medical imaging modality wherein the lumen at each location of the structure can be delineated in a two-dimensional view with a segmentation software. At step 120, segmentation software acquires data including voxel count, voxel dimensions, volume, and/or surface area. The segmentation software must be capable of delineating the lumen by filling the lumen area with a labeling tool that provides statistics such as voxel count, volume, and/or surface area. As shown in FIG. 1, at step 130, the cross-sectional area at each location is found by delineating the cross-sectional areas of the lumen using segmentation software.

Two methods may be used for collecting the cross-sectional area data. First, all the anatomical structures from a population may be scaled so they are the same size, and then cross-sectional data is collected in discrete, even increments. Second (in the alternative), the original scale for all anatomical structures in a population may be kept, and cross-sectional area data may be collected at relative intervals (as a percentage). To further illustrate this point, a plurality of locations may either be discrete and even relative distances of a dimension of the anatomical structure (e.g., every 5 slices on a sagittal, transverse, or frontal plane, given the same slice thickness on CT imaging), or a percentage of the distance from one end of the structure to the other (such as 25%, 50%, 75% and 100% of the length of the anatomical structure).

In an example of the relative method in a candidate canine nasopharynx model, the cross-sectional area of the lumen of the nasopharynx is determined on a transverse view with a labelling tool using segmentation software at the start of the soft palate (0%), at 25% of the distance from the starting point on one end of the structure to the other, at 50% of the distance from the starting point on one end of the structure to the other, and at 75% of the distance from the starting point on one end of the structure to the other. As many starting points as desired may be included in the calculations. Data for the relative cross-sectional area of the nasopharynx is determined by the ratio of the cross-sectional area at one location, to another location, repeated for all four location combinations. This provides a plurality of data points for each subject.

Using the cross-sectional areas determined in step 130, population data is then determined by taking the ratio of the cross-sectional area at one location to a different location, repeated for all possible location combinations at step 140. Each location combination represents a data category (e.g., CSAlocation1/CSAlocation2=category 1, CSAlocation2/CSAlocation3=category 2 . . . , etc.). At step 150, a sum of squares statistical method is used at each category, and then summed across all categories, in every subject in a subject population to find the subject with the least cumulative variability from the average. This subject becomes the optimal subject in a population.

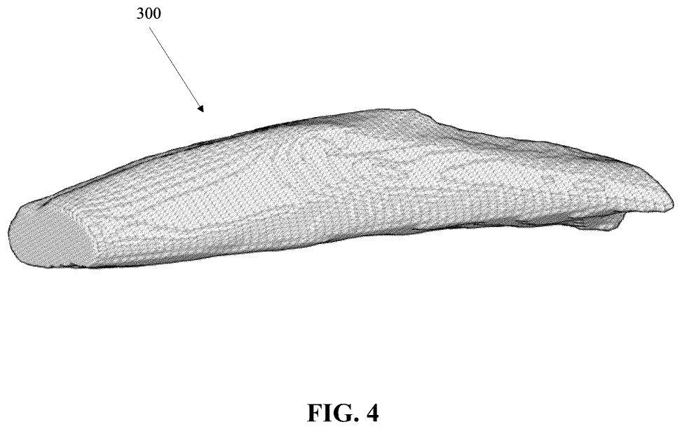

Once an optimal subject is determined at step 150, the three-dimensional model of the area of interest of that optimal subject becomes the candidate model 300 for the idealized implant. The optimal subject refers to the subject in the population that has the anatomical structure that is the most representative of the average shape of the structure. An exemplary candidate model 300 of a candidate canine nasopharyngeal model is shown in FIG. 4. The candidate model 300 was segmented from the nasopharynx of the optimal subject in that population.

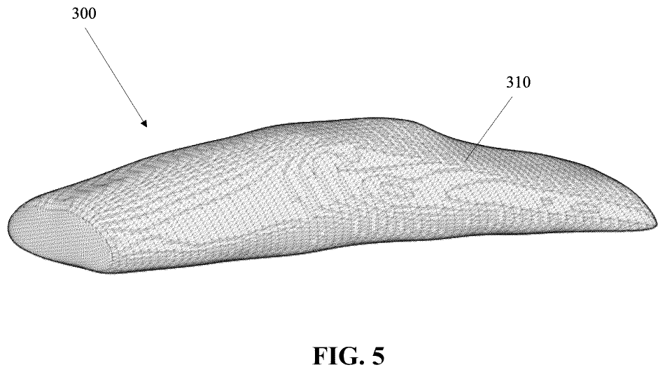

Such a candidate model 300 is then used in step 160, where the candidate model is refined, and the idealized implant shape is constructed. Using computer aided design software, the candidate model 300 is refined by smoothing the edges. As shown in FIG. 5, after refining, a candidate model 300 has smooth edges 310. The candidate model 300 is then shelled to provide a hollowed cavity, resulting in an idealized implant model 305. As seen in FIG. 5, the idealized implant model 305 has a hollow internal cavity 320. The idealized implant 330 is then constructed via thickening the wall of the hollowed cavity by building inwards or outwards from the virtual model of the idealized implant model 305. At step 170, the idealized implant 330 may then be constructed either through injection molding or through direct construction, e.g., through 3D printing. The idealized implant 330 may be produced with a physical material as a physical product. Such physical materials include, but are not limited to, artificial materials such as implantable metals or polymers, and biological materials.

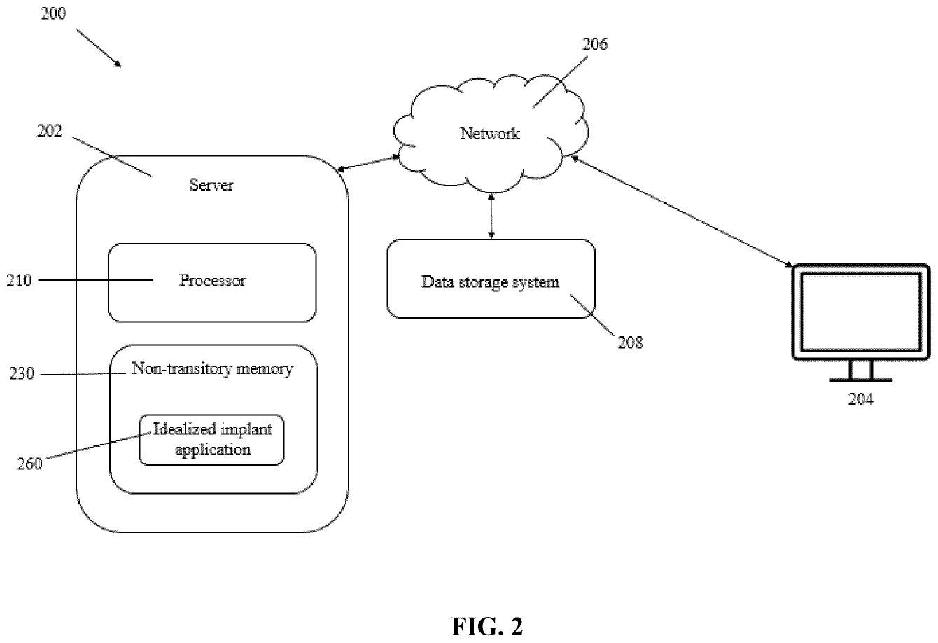

The aforementioned method 100 for designing and fabricating an idealized implant for a hollowed anatomical structure comprises obtaining a plurality of images captured from a subject population may be implemented according to an embodiment in a computing system 200, comprising a server 202, a client device 204, a network 206, and data storage system 208. Some or all of method 100 may be performed by any suitable system by various components of the system 200 described in conjunction with FIG. 2, such as the server 202, by multiple computing devices in a distributed system of a computing resource service provider, or by any electronic client device such as the client device 204.

The server 202 may comprise a processor 210 and a non-transitory memory 230. The non-transitory memory 230 may include an idealized implant application 260 stored therein, that when executed by the processor 210, causes the server 202 to perform acts associated with the idealized implant application 260. According to an embodiment, the system 200 includes a client device 204, which includes any appropriate device operable to send and/or receive requests, messages, or information over an appropriate network 206 and convey information back to a user of the device. The idealized implant application 260 may have both server-side and client device-side functionality. The client device 204 comprises appropriate elements such as a non-transitory memory and a processor that can execute computer-readable instructions (e.g., a client device 204 processor may execute functions of the idealized implant application 260). According to an embodiment, the server 202 and the client device 204 may be connected via network 206 to data storage 208.

The data storage 208 may contain patient information including imaging data, and population data including demographic information. The system 200 receives user input in the form of patient information (including imaging data) and population information (including demographic information) from an input device 250. According to various embodiments, the computing system 200 may be comprised of one stand-alone device or a plurality of devices, and the system 200 may communicate with one or more external devices.

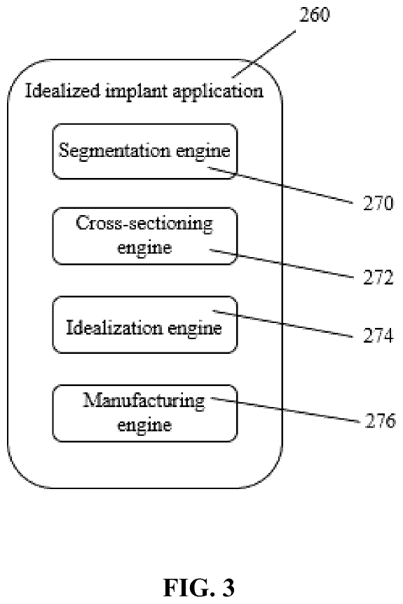

According to an embodiment as shown in FIGS. 2 and 3, the idealized implant application 260 stored within the non-transitory memory 220 comprises a segmentation engine 270, a cross-sectioning engine 272, an idealization engine 274, and a manufacturing engine 276. As used herein, each operation performed by the segmentation engine 270, the cross-sectioning engine 272, the idealization engine 274, and/or the manufacturing engine 276 may also be described as being performed by the idealized implant application 260. Using the segmentation engine 270, a user is able to utilize imaging data and select a plurality of locations within a three-dimensional model of an area of interest and, using the cross-sectioning engine 272, generate a corresponding plurality of cross-sectional areas for the plurality of locations via a graphic user interface 240. The optimization engine 274 enables a user to determine an idealized implant model 305 based on cross-sectional data from a population and demographic information. Last, the manufacturing engine 276 utilizes computer aided design software to construct idealized implants 330.

According to an embodiment of the invention, a negative space inside a mold represents the shape of the idealized implant shape along with at least one venthole and an injection port. The idealized implant 330 presents a surface that can be in contact with an area of the anatomical structure to provide support. In an example, the idealized implant 330 generated by the manufacturing engine 276 can be hollow and/or solid and takes up space within part or all of the anatomical structure. The idealized implant 330 may be irregularly shaped and can be concave and/or convex, as it represents the form of at least some part of the anatomical structure for which it is intended. The thickness of the idealized implant 330 may vary depending on the hardness of the implant material used, the forces applied to the implant when situated in the anatomical structure, and the dynamic movement of the anatomical structure.

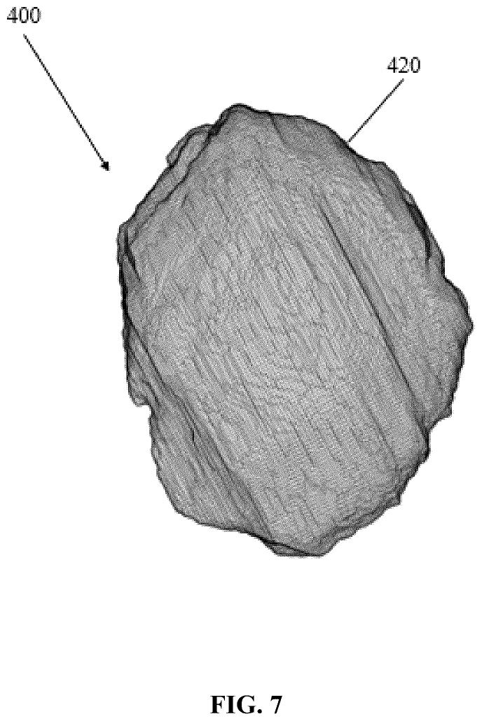

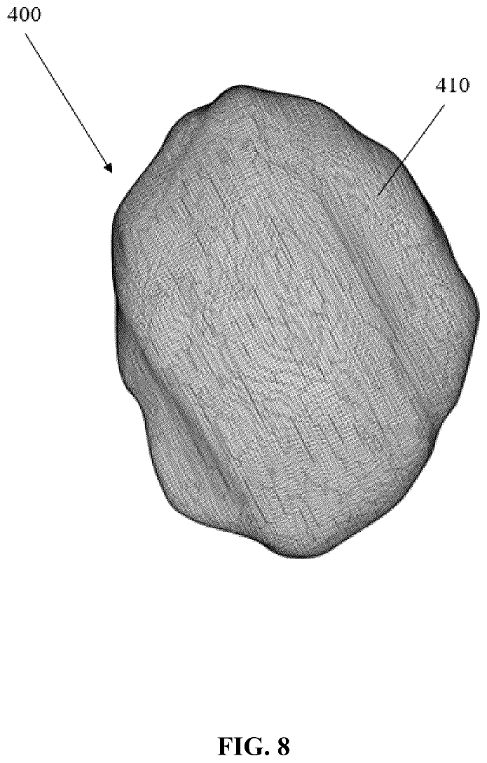

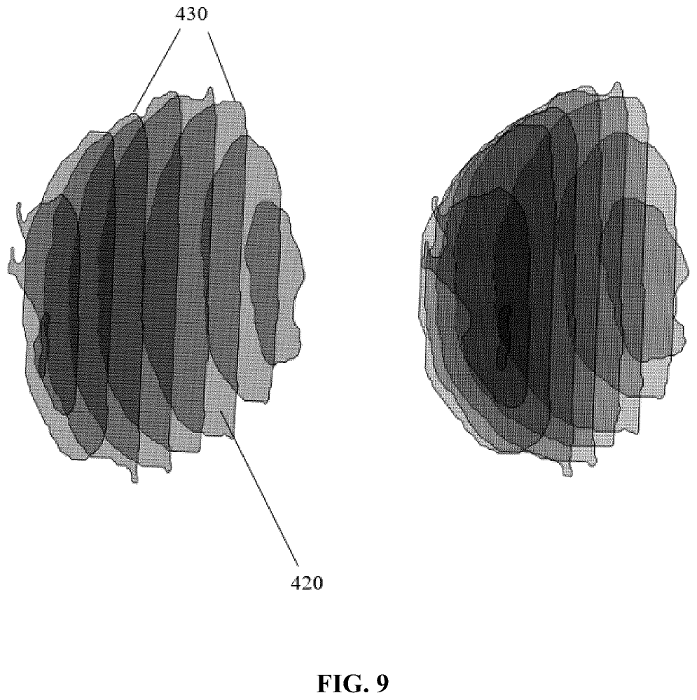

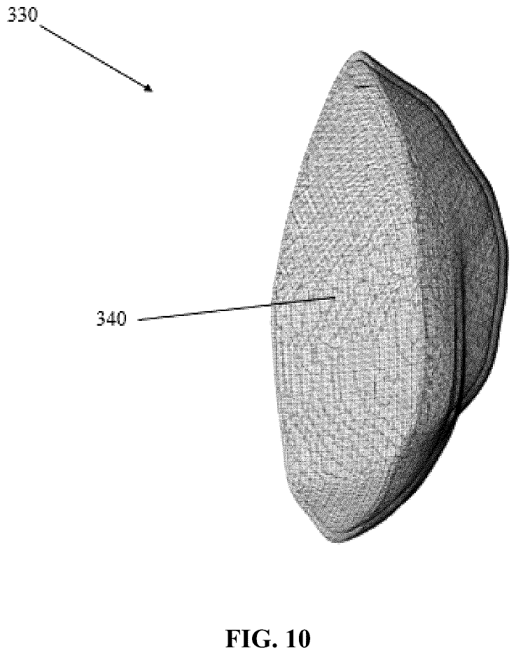

As seen in FIGS. 7 and 8, a candidate non-tubular structure model 400 may also be refined with computer aided design software by creating smooth edges 410. As shown in FIGS. 7 to 9, the shape of an anatomical structure is characterized by the relative cross-sectional area 430 of the lumen 420 at a plurality of discrete or relative locations 440 in the hollowed anatomical structure. According to an embodiment of the invention, the method of segmentation and taking the relative cross-sectional areas 430 of the lumen 420 at a plurality of discrete or relative locations 440 contained in the segmentation engine 270 and the cross-sectioning engine 272 could be utilized for a closed structure that is not open at one or either end such as a cavity (heart, lungs etc.). The idealized implant 330 can provide internal support to any hollowed anatomical structure (e.g., heart, lungs, skull, airways, blood vessels). The method of segmentation and taking the relative cross-sectional areas 430 of the lumen 420 at a plurality of discrete or relative locations 440 may be utilized for any hollowed anatomical structures (tubular or non-tubular), including those which are closed at one or either end. According to an embodiment as seen in FIG. 10, an idealized implant 330 has a hollow internal cavity 340 and can be cropped for a section of a hollowed anatomical structure and not used in its entirety.

The present embodiment(s) may be a system, a method, and/or a computer program product. The computer program product may include a computer readable storage medium (or media) having computer readable program instructions thereon for causing a processor to carry out aspects of the present embodiment(s).

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Accordingly, a computer readable storage medium, as used herein, is non-transitory.

Computer readable program instructions described herein can be downloaded to respective computing/processing devices from a computer readable storage medium or to an external computer or external storage device via a network 206, for example, the Internet, a local area network, a wide area network and/or a wireless network. The network 206 may comprise copper transmission cables, optical transmission fibers, wireless transmission, routers, firewalls, switches, gateway computers and/or edge servers. A network adapter card or network interface in each computing/processing device receives computer readable program instructions from the network 206 and forwards the computer readable program instructions for storage in a computer readable storage medium within the respective computing/processing device.

Computer readable program instructions for carrying out operations of the present embodiment(s) may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present embodiment(s).

Aspects of the present embodiment(s) are described herein with reference to flowchart illustrations and/or block diagrams of methods, apparatus (systems), and computer program products. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine (such as a client device 204), such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to the various embodiments. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The various embodiments of the present invention described herein offer a benefit over generic implants, because they are capable of representing the optimal shape of a population. For example, in an airway, medical literature shows that a poor implant fit is linked to a high prevalence of complications, including migration, infection and fistula development, ultimately leading to a poor prognosis. By fitting optimally to the relevant hollowed anatomical structure 400, the idealized implant 330 will better fit and maintain pressure with the lumen 420 of the hollowed anatomical structure 400 and facilitate faster and better healing. The various embodiments of the present invention also offer a benefit over traditional custom implants and methods. Traditional custom implant methods include long wait times and high costs in order to produce a custom implant for a particular patient. This presents a significant barrier to a lot of patients in need of medical implants. This leaves patients stuck between generic, ill-fitting implants which are fast and relatively inexpensive, and custom, expensive implants which take a long time to produce. Embodiments of the present invention offers a solution to these problems and offers patients a method for producing idealized implants 330 which are affordable, offer a curated fit, and can be rapidly manufactured.

Further, the various embodiments of the present invention offer a benefit over any other optimized implant production methods available by offering a method for design and production of idealized implants 330 which can be applied to both tubular and non-tubular structures, as well as to hollowed anatomical structures which are closed at one or either end such as a cavity (heart, lungs etc.). As such, these idealized implants 330 can be used to treat the obstruction of any anatomical structure. The versatility, optimization, speed, and affordability of the embodiments of the present invention will improve the quality of patient treatments, reduce complications, and improve patient outcomes.

Various embodiments of the invention have been described in detail. The present invention may be embodied in other specific forms without departing from the essential characteristics thereof. Certain adaptations and modifications of the invention will be obvious to those skilled in the art. Therefore, the presently discussed embodiments are considered to be illustrative and not restrictive, the scope of the invention being indicated by the appended claims rather than the foregoing description and all changes which come within the meaning and range of equivalency of the claims are therefore intended to be embraced therein.

We claim:

1. A computing system for generating implant designs, comprising:
a processor; and
a non-transitory memory storing an idealized implant application comprising a segmentation engine, a cross-sectioning engine, and idealization engine, and a manufacturing engine, that, when executed by the processor, cause the processor to perform acts comprising:
obtaining a plurality of images captured from a subject population with a normal hollowed anatomical structure via an input device;
segmenting at least one region of interest in each of the plurality of images to provide a three-dimensional model of an area of interest via segmentation software that provides data;
selecting a plurality of locations within each three-dimensional model of an area of interest and generating a corresponding plurality of cross-sectional areas for the plurality of locations via a graphic user interface;
determining population data from a plurality of data points that represent the relative shape of the hollowed anatomical structure for each subject in a population by determining the ratio of the cross-sectional area at one location to a different location, repeated for all possible location combinations, wherein each location combination represents a data category;
discovering an optimal subject in a population from population data by statistical methods; and
utilizing a model generator to refine a three-dimensional model of the optimal subject and design an implant shape corresponding to the optimal subject in a population.

2. The computing system of claim 1, wherein a population includes any grouping of subjects that have a common characteristic which distinguishes them from other groups of subjects.

3. The computing system of claim 1, wherein a plurality of locations comprises discrete and even, or relative distances of a dimension of the anatomical structure.

4. The computing system of claim 1, wherein the data provided by the segmentation software includes surface area, volume, voxel dimensions, and voxel count.

5. The computing system of claim 4, wherein the plurality of cross-sectional areas are determined either directly from acquiring surface area or indirectly from the relationship between voxel count or volume and voxel dimensions.

6. The computing system of claim 1, wherein refining the three-dimensional model of an optimal subject includes changing the angularity of the edges of the candidate structure model using computer aided design software by using a smoothing tool or feature.

7. The computing system of claim 1, wherein discovering an optimal subject includes determining the average for each data category across the population data.

8. The computing system of claim 7, wherein discovering an optimal subject includes using a sum of squares method at each data category, then summing across all data categories, in every subject to find the subject with the least cumulative variability from the average.

9. The computing system of claim 1, wherein designing the implant shape may include cropping a candidate structure model to a section of a concave/convex structure and hollowing the candidate structure model to a particular wall thickness using computer aided design software.

10. A method for designing and manufacturing implants, the method comprising:
obtaining a plurality of images captured from a subject population with a normal hollowed anatomical structure;
segmenting at least one region of interest in each of the plurality of images to provide a three-dimensional model via segmentation software that provides data;
selecting a plurality of locations within each three-dimensional model of a hollowed anatomical structure and generating a corresponding plurality of cross-sectional areas for the plurality of locations via a graphic user interface;
determining population data from a plurality of data points that represent the relative shape of the hollowed anatomical structure for each subject in a population, wherein each location combination represents a data category;
discovering an optimal subject in a population from population data by statistical methods;
utilizing a model generator to refine and construct an idealized implant shape corresponding to the optimal subject in a population; and
manufacturing the idealized implant shape.

11. The method of claim 10, wherein a population includes any grouping of subjects that have a common characteristic which distinguishes them from other groups of subjects.

12. The method of claim 10, wherein a plurality of locations comprises discrete and even, or relative distances of a dimension of the anatomical structure.

13. The method of claim 10, wherein the data provided by the segmentation software includes surface area, volume, voxel dimensions, and voxel count.

14. The method of claim 13, wherein the plurality of cross-sectional areas are determined either directly from acquiring surface area or indirectly from the relationship between voxel count or volume and voxel dimensions.

15. The method of claim 10, wherein refining the three-dimensional model of an optimal subject includes changing the angularity of the edges of the candidate structure model using computer aided design software by using a smoothing tool or feature.

16. The method of claim 10, wherein discovering an optimal subject includes determining the average for each data category across the population data.

17. The method of claim 16, wherein discovering an optimal subject includes using a sum of squares method at each category, then summing across all categories, in every subject to find the subject with the least cumulative variability from the average.

18. The method of claim 10, wherein in designing and constructing an idealized implant shape may include cropping a candidate structure model to a section of a concave/convex structure and hollowing a candidate structure model to a particular wall thickness using computer aided design software.

19. The method of claim 10, wherein manufacturing may occur through direct construction through 3D-printing from computer aided design software or through injection molding manufacturing, among other manufacturing methods, wherein a negative space inside a mold for injection molding manufacturing represents the shape of the idealized implant shape along with at least one venthole and an injection port.

20. The method of claim 10, wherein the manufacturing of an idealized implant may utilize artificial and/or biological materials.
